(19) United States
(12) Patent Application Publication
(10) Pub. No.: US 2005/0232944 A1
(43) Pub. Date: Oct. 20, 2005

(54) Composition and paste for eliminating abnormal conditions in a human affected by chaotic environmental electromagnetism and the manufacturing method thereof

(76) Inventors: Serena Hui-Yee Hsu, Melbourne (AU); Ting-Jung Lin, Yanshuei Township (TW)

Correspondence Address: HERSHKOVITZ & ASSOCIATES, 1725 I STREET NW, SUITE 300, WASHINGTON, DC, 20006, US

(21) Appl. No.: 10/916,529

(22) Filed: Aug. 12, 2004

(30) Foreign Application Priority Data
Apr. 15, 2004 (TW) 093110477

(51) Int. Cl. A61K 35/80

(52) U.S. Cl. 424/195.17

(57) ABSTRACT

The present disclosure is related to a composition having an effective amount of algae for eliminating abnormal conditions in a human affected by chaotic environmental electromagnetism. Furthermore, the present invention is also related to a paste having an adhesive layer and a non-adhesive layer formed from the composition for eliminating abnormal conditions in a human affected by chaotic environmental electromagnetism as described above. The present invention is also related to methods for manufacturing the paste and the composition as described above. In addition, the present invention is related to a method of eliminating abnormal conditions in a human affected by electromagnetism.

### BACKGROUND OF THE INVENTION

1. Field of the Invention

The present invention relates to a composition for eliminating abnormal conditions in humans affected by chaotic environmental electromagnetism and the manufacturing method, and more particularly to a paste for eliminating abnormal conditions in humans affected by chaotic environmental electromagnetism and the manufacturing method thereof.

2. Description of Related Art

In this 21^{st} century, humans are enjoying the advanced civilization in modern science and technology. According to our advanced progression in medicine and the development in science and technology, the lifespan of humans has generally increased.

Currently, cell phones and computers have become important necessities for many people throughout the world. However, despite numerous medical and scientific investigations on the harmful influences of cell phones and computers on human health, there is another group of scientists and cell phones manufacturing companies who claim that cell phones and computers are totally harmless. Hence, such endless debatable issue on whether cell phones and computers have any harmful influential effects on human health then becomes a problem.

Researchers inferred that interference from electromagnetism might lead to the possibility of diseases. Some reports indicated that exposing in vitro human large intestine cancer cells to an electromagnetized environment would cause these cancer cells to proliferate excessively; and the proliferation rate was more rapid compared to in vitro cancer cells in a non-electromagnetized environment. Furthermore, cancer cells cultured in an environment interfered with by electromagnetism would become more resistant to the human immune cells. In an environment interfered with by electromagnetism, the human nature killer cells (NK cells—normally activated to defend against tumors) clearly exhibit structural changes.

Other studies reported that certain diseases were related to electromagnetic exposure so the interference on human physiology affected by exposure to electromagnetism must be avoided. However, there is nothing available to date that can effectively prevent such interference caused by electromagnetism. Especially in our era where we are almost totally dependent upon electronic devices such as cell phones, computers, televisions, it is very difficult to avoid prolonged exposure to electromagnetism emitted by these devices. Although there are various inventions utilizing inorganic physical materials to shield users from electromagnetism, none of these materials are able to achieve their anticipated beneficial health effects. Researchers have already proven that usage of computers and cell phones are the most severe types of electromagnetic interference. Medical experiments had proven that when a person underwent a brain computed tomography scan while operating computer terminals/monitors or communicating on a cell phone, an abnormal phenomenon would be exhibited on the brain computed tomography. Yet, computers and cell phones have become an important part of our lives. Therefore, the vitally crucial factor then lies upon how to efficaciously prevent these negative influential damages from electromagnetism on our health.

### SUMMARY OF THE INVENTION

An aspect of the present invention is to provide a composition comprising an effective amount of algae for eliminating abnormal conditions in a human affected by chaotic environmental electromagnetism.

Another aspect of the present invention is to provide a paste comprising an adhesive layer; and a non-adhesive layer comprising the composition according to the present invention. The paste according to the present invention is provided for eliminating abnormal conditions in a human affected by chaotic environmental electromagnetism as described above.

Still another aspect of the present invention is to provide a method for manufacturing the composition according to the present invention. The method comprises providing algae; and preparing the algae to a composition.

Still another aspect of the present invention is to provide a method for manufacturing the paste as described above comprising providing algae; using the algae to prepare a non-adhesive layer; combining an adhesive layer and the non-adhesive layer; and obtaining a paste.

Still another aspect of the present invention is to provide a method of eliminating abnormal conditions in a human affected by electromagnetism, comprising providing a composition containing algae; and applying the composition to a source generating electromagnetism.

Preferably, the composition was prepared in the form of a gel, a paste, a spray, or immobilized in/into a solid. More preferably, the composition was a paste.

Preferably, the algae are Chlorella, Spirulina, Haematococcus or Dunaliella. More preferably, the algae are Dunaliella.

Further benefits and advantages of the present invention will become more apparent upon careful reading of the detailed description.

### DETAILED DESCRIPTION OF THE INVENTION

A preferred embodiment in accordance with the present invention is related to a composition for eliminating abnormal conditions in a human affected by chaotic environmental electromagnetism. The composition comprises an effective amount of algae. The composition may be prepared into any shapes or forms, such as a gel, a paste, a spray or be immobilized in/into a solid. The algae capable of photosynthesizing may be of any general such as Chlorella, Spirulina, Haematococcus or Dunaliella. Preferably, the algae are Chlorella. In a preferred embodiment of the present invention, the algae may be extracted and in the form of a concentrated extract.

The inventors have performed experiments and research in order to fully understand the key problem of cell phones, computers and electronic devices on human health. Finally, the results show that the actual problem does not lie upon whether cell phones, computers and electronic devices have any influential effects on human health; instead the key critical factor that influences human health is derived from the SIM card and all mass media storages embedded within the cell phones, computers and electronic devices. Once the cell phone is fitted with the SIM card (including PHS system or phones that do not need SIM cards), it immediately starts to influence our human physiological functions, which can be demonstrated via the “Single Muscle Evaluation” test. However, any cell phone that does not have a SIM card fitted inside (except the PHS system or phones that do not utilize SIM cards or mass media storages) will not demonstrate such phenomenon on our human physiological functions. Such influential phenomena from a SIM card embedded cell phones and all mass media storages not only exhibit on human physiology, but also apparent in many animals and cellular experiments. Nevertheless, these data and results have not been able to allow users of cell phones, computers and electronic devices to vividly feel these influential phenomena.

It is understood from the above that the harmful influences on human physiological functions from using cell phones, computers and electronic devices actually derive from SIM cards and all mass media storages, and not literally derive from cell phones, computers and electronic devices themselves. The present invention has demonstrated that such harmful health phenomena derive from the electrical potential energy exchange of information between the SIM card and cell phone (or between mass media storages and electronic devices, eg computers, PDA), thus resulting in an electromagnetical interference of bioenergetics.

The present invention shows that once a SIM card is fitted into cell phones (and/or mass media storages inserted into electronic devices), there will be an immediate interference on the normal physiological functions of bioenergetics in humans and organisms; and such phenomena is absent when the SIM card is not fitted into cell phones (and/or mass media storages not inserted into electronic devices). The influential effects on human physiological functions of bioenergetics can be demonstrated on the central nervous system using the “Single Muscle Evaluation” test. The function of the central nervous system is evaluated by first using a cell phone with and secondly without the SIM card inside to perform the “Single Muscle Evaluation” test for muscle strength. A cell phone with a SIM card interferes with the normal function of the central nervous system presenting its inability to control the strength of a single muscle, that is, a weakness in muscle strength is exhibited. In addition, when the user is talking on a cell phone, the user's blood pressure increases and the heart rate decreases. Thus, in order to efficaciously protect human health from these possible harmful effects generated by cell phones and computers, it is essential to target these problems associated with the use of the SIM cards and all mass media storages.

Since natural chlorophyll is the best electron transducer in organisms, natural chlorophyll is capable of balancing out any chaotic biological electron disarrangement so as to achieve equilibrium. Injuries or damage to organisms due to interference caused by electromagnetism, are in fact the result of interference to the equilibrium of cellular electrons, e.g. free radical damages. Hence, various kinds of chlorophyll are examined, in light of determining certain materials that are able to efficaciously eliminate and restore any abnormal physiological phenomena generated by those electronic devices such as computers and cell phones.

One aspect of this invention, a more specific factor, is a composition that can eliminate any abnormal human physiological phenomena generated by electromagnetism. The composition comprises an effective amount of algae that can eliminate any abnormal human physiological phenomena generated by electromagnetism. The composition can be prepared in different shapes and forms, such as glue, paste, spray or immobilized into a solid body. The algae with ability to photosynthesize may be of any genera, such as Chlorella, Spirulina, Haematococcus or Dunaliella. Preferably, the algae are Chlorella.

On the other hand, the algae may be an extract and in the form of a concentrated extract.

The composition can be manufactured in different shapes and forms. The composition can be laid on, pasted on or affixed onto a SIM card or a mass media storage to convert and transform the energy produced by the interaction between the SIM card and the cell phone or between a mass media storage and any other devices that produce electromagnetism. Hence, the composition can be used for different models, functions, and structures of cell phones or any electronic devices. Due to different diversified functions of cell phones and electronic devices and the different duration of talk-time, it is necessary to change the composition or paste from time to time. Since the composition is organic, it has a lifespan that starts upon usage. It is recommended that the composition be changed about every two weeks to three months in order to achieve its effective function in eliminating any abnormal human physiological phenomena generated by electromagnetism.

In a preferred embodiment in accordance with the present invention, a purified algae culture in the form of concentrated extract is molded into a composition that enables it to be affixed into the cell phone so that it is in contact with the SIM card; which then enables this composition to effectively prevent harmful influences on a human physiology generated by cell phones, computers and other electronic devices. The present invention has found that the algae effective in achieving such results includes Chlorella, Spirulina, Haematococcus, Dunaliella and/or algae capable of photosynthesizing; with Chlorella having the most effective function amongst the algae family.

A preferred embodiment in accordance with the present invention is to provide algae via different methods and to culture the algae under various different conditions, such as culturing in an electromagnetized environment or a normal non-electromagnetized environment. Since the method of purifying the algae is well known in the art it is not further described.

There are various different effective methods in obtaining algae in the form of concentrated extract which is well known amongst the skills in the art. One method for providing concentrated extracting algae comprises:

(1) washing and centrifuging purified algae culture to form an algae suspension of a suitable concentration;

(2) spray drying (or freeze drying) the algae suspension to form powder;

(3) adding water to the powder and heating;

(4) filtering the algae from the heated suspension; and

(5) drying the filtered suspension to obtain a concentrated extract.

In another aspect, a preferred embodiment in accordance with the present invention provides a method for manufacturing a paste for eliminating abnormal condition in human affected by chaotic environmental electromagnetism comprising:

providing algae;

using the algae to prepare a non-adhesive layer;

combining an adhesive layer and the non-adhesive layer; and

obtaining a paste.

Preferably, the algae may be Chlorella, Spirulina, Haematococcus or Dunaliella.

In a preferred embodiment in accordance with the present invention, a concentrated algae extract is applied on a SIM card during affixation onto the cell phone so that it can effectively prevent any possible harmful influences on human physiology generated by cell phones. The “Single Muscle Evaluation” test demonstrates a normal central nervous system whereby the ability in muscle control is affected. Measurement of blood pressure and heart rate while user is talking on a cell phone demonstrates that interference no longer exists as compared to use of a cell phone not fitted with the composition on the SIM card.

In a preferred embodiment of the present invention, there are three tests to allow cell phone users to vividly testify and feel the severe influential effects of cell phones fitted with SIM cards, computers or any electronic devices on the physiological phenomena. Firstly, measurement of blood pressure (a mercury sphygmomanometer is used to avoid any possible interference) when a person is talking on a cell phone or operating a computer demonstrates a phenomenon of an increased blood pressure in comparison to a person not talking on a cell phone or not operating a computer. Secondly, measurement of heart rate under the same conditions demonstrates a phenomenon of a decreased heart rate. Thirdly, performing the “Single Muscle Evaluation” test under the same condition demonstrates a phenomenon whereby the central nervous system exhibits a decrease in muscle strength.

This invention utilizes the “Single Muscle Evaluation” test as an effective testing protocol that monitors the central nervous system in its function to transmit information between the brain and the muscle. Under normal circumstances (that are without interference caused by electromagnetism), the central nervous system is capable of function normally in its transmission of messages from the brain to the muscle, thus exhibiting normal muscle strength. However, once the user holds a cell phone with a SIM card inside, the transmission of messages from the brain to the muscle is interfered with; thus exhibiting a weakness in muscle strength that reflects such interference on the central nervous system causing its inability to function normally.

The procedure of the “Single Muscle Evaluation” test is described as follows: a subject performing the test is called the Tester, while a subject who is being tested is called the Testee.

Made sure that the Tester and Testee were not carrying any cell phones or any electronic devices on them before performing this test. Asked the Testee to sit up straight or stand upright without slouching. The Testee then stretched out one arm either to the side or directly in front (making sure that the arm was straight and horizontal to the ground). Next, the Tester supported the Testee's shoulder with one hand. The other hand of the Tester exerted pressure on the forearm just above the wrist; while the Testee had to resist this pressure without changing hand or body position. Under normal circumstances, the Testee was able to resist this pressure (provided there was no previous injury or spinal misalignment). Next asked the Testee to hold a cell phone with the SIM card inside. Repeating this test resulted in an obvious weakening of muscle strength. Next, the test was performed again without the Testee holding a cell phone and the muscle strength was regained immediately.

In the quest for an effective method in preventing the influences of electromagnetism created by cell phones, computers and electronic devices on human physiological functions, the inventor performed extensive research that showed the efficacy of this composition. A preferred embodiment of the present invention provides a composition comprising algae and/or photosynthetic algae that enable to contact with the SIM card during affixation onto the cell phone so that it can effectively prevent any possible harmful influences on the human physiology generated by cell phones etc. The efficacy of this composition is measured by the “Single Muscle Evaluation” test that reflects a normal central nervous system in its ability of muscle control; measuring of blood pressure indicates no interference with a normalization of blood pressure value; and, measuring of heart rate shows a normal value indicating no interference.

The term “algae” as used herein refers to any kind of algae, such as a concentrate, a powder, a solution, or a suspension.

The term “a source generating electomagnetism” as used herein refers to electronic devices that generates electromagnetism, such as cell phones, computers and monitors.

The term “chaotic environmental electromagnetism” as used herein refers to a disorganized or disarrayed electromagnetism or microwaves in the environment inside or outside of an organism. Electromagnetism exists everywhere around us, just like the Earth itself has a huge electromagnetic field. However, it is only the disorganization or disarray type of electromagnetism that has a role in affecting organisms. To illustrate, the central nervous system functions via its transmission of messages from the brain to all parts of the body. Its transmission of messages works via nerve propagation and nerve impulses which can be easily interrupted by chaotic environmental electromagnetism internally or externally. The “Single Muscle Evaluation” test represents incapability of the central nervous system to transmit messages to contract the muscle, thus resulting in a weakening in muscle strength. Yet, without the influence by chaotic environmental electromagnetism, the central nervous system is capable of transmitting messages to contract the muscle, thus presenting normal muscle strength.

The term “abnormal condition in humans” as used herein refers to any changes to normal condition, such as abnormal physiological functions leading to abnormal conditions, signs or symptoms. The example showed a single muscle affected by chaotic environmental electromagnetism in its muscle strength. One vital organ in humans is the heart and it is composed of cardiac muscles. Under the influences of such chaotic environmental electromagnetism, the cardiac muscles present a weakening in strength, due to the incapability of the central nervous system to transmit messages normally. Hence, with a weakening of cardiac muscles and a demand for a certain volume of blood to be pumped out each heart beat, an abnormal condition arises. The results represent an increase in blood pressure and a decrease in heart rate. This abnormal condition appears not because the person has a predisposing disease such as hypertension but as a result of an influential effect from chaotic environmental electromagnetism on the normal human physiology. Such abnormal condition can be reversed when the influencing factor (chaotic environmental electromagnetism) is removed. However, long term exposure to such chaotic environmental electromagnetism can cause severe tissue damage and permanent physiological changes that in turn leads to disease(s).

The term “abnormal conditions in humans affected by chaotic environmental electromagnetism” as used herein refers to internal or external chaotic environmental criteria of electromagnetism or microwaves that have detrimental health effects or damage on organisms over time.

The characteristics, qualities and advantages of this invention are further illustrated in the following examples.

### EXAMPLES

The following examples are used to illustrate this present invention for further description. These examples do not, under any circumstances, restrict the scope of patent, but indicate how to enforce the materials and method of this invention.

### Example 1

Effect of the Composition Comprising Algae Coated on a SIM Card Inside, the Card Being Received in a Cell Phone

1.1 Preparation of a Composition for Eliminating Abnormal Conditions in a Human

Algae was provided through purification culture and manufactured into a paste form.

1.2 Blood Pressure Test

The paste was prepared as described in example 1.1 and the paste was affixed onto the SIM card inside the cell phone. A mercury sphygmomanometer was used to measure the blood pressure of the Testee. The blood pressure was taken under normal circumstances without any cell phone or electronic devices referred as control. The blood pressures were measured under the Testee talking on the cell phone without the paste on the SIM card and the Testee talking on the cell phone with the paste on the SIM card. The results showed that the Testee talking on the cell phone with the paste on the SIM card had the same blood pressure as the control (Testee not using or holding a cell phone with a SIM card or holding any electronic devices). Also, it was found that the Testee had an increase in blood pressure without the affixation of the paste.

1.3 Heart Rate Test

The paste was prepared as described in example 1.1 and the paste was affixed onto the SIM card inside the cell phone. A heart rate monitor was used to measure the heart rate. The heart rate was taken under normal circumstances without any cell phone or electronic devices referred as control. The heart rates were measured under the Testee talking on the cell phone without the paste on the SIM card and the Testee talking on the cell phone with the paste on the SIM card. The results showed that the Testee talking on the cell phone with the paste on the SIM card had the same heart rate as the control (Testee not using or holding a cell phone with a SIM card or holding any electronic devices). Also, it was found that the Testee had an increase in heart rate without the affixation of the paste.

1.4 “Single Muscle Evaluation” Test

The paste was prepared as described in example 1.1 and the paste was affixed onto the SIM card inside the cell phone. The “Single Muscle Evaluation” test was used to measure the muscle strength. The test was performed with the Testee sitting up straight or standing upright without slouching. The Testee stretched out one arm either to the side or directly in front, making sure that Testee's arm was horizontal to the ground. A Tester supported the Testee's shoulder of the out-stretched arm with one hand, while the other hand of the Tester exerted pressure on the forearm just above the wrist. The Testee resisted this pressure exerted by the Tester without any postural change. This was because any postural change made by the Testee altered the strength of the muscle due to muscle requirement to increase strength, thus giving a false positive result.

The control was the muscle strength of the Testee taken under normal circumstances without any cell phone or electronic devices (provided the Testee had no previous injury or spinal misalignment that may affect the accuracy of the reading). It was found that the Testee talking on the cell phone without the affixation of the paste showed a weakness in muscle strength, indicating an inability of the Testee to resist the exerted pressure. Further, when the Testee talking on the cell phone with the affixation of the paste showed normal muscle strength as the control. That was, the Testee had the ability to resist the exerted pressure or had the same strength in muscle resistance under holding the paste affixed on the SIM card.

The invention has been explained in relation to its preferred embodiment, nevertheless many other possible modifications and variations can be made without departing from the spirit and scope of the invention as hereinafter claimed. Although the present invention accounts for the specified facts, however, it must be understood that the present invention will not be restricted to just the facts or factors specified herein. In actuality, the aspect on the enforcement of the present invention and described methodology within, inclusive of any different modifications or variations are all included within the listed scope of patent application.

1. A composition comprising an effective amount of algae for eliminating abnormal conditions in a human affected by electromagnetism.

2. The composition as claimed in claim 1 prepared in the form of a gel, a paste, a spray, or immobilized in/into a solid.

3. The composition as claimed in claim 2, wherein the algae are Chlorella, Spirulina, Haematococcus, or Dunaliella and/or the algae with ability to photosynthesize.

4. The composition as claimed in claim 3, wherein the algae are Chlorella.

5. The composition as claimed in claim 1, wherein the algae are in the form of a concentrated extract.

6. A paste, comprising:
an adhesive layer; and
a non-adhesive layer comprising the composition as claimed in claim 1 for eliminating abnormal conditions in a human affected by electromagnetism.

7. The paste as claimed in claim 6, wherein the algae are Chlorella, Spirulina, Haematococcus or Dunaliella.

8. The paste as claimed in claim 7, wherein the algae are Chlorella.

9. The paste as claimed in claim 6, wherein the algae are in the form of a concentrated extract.

10. A method for manufacturing the paste as claimed in claim 6 comprising:
providing algae;
using the algae to prepare a non-adhesive layer;
combining the adhesive layer and the non-adhesive layer; and
obtaining a paste.

11. The method as claimed in claim 10, wherein the algae are in the form of a concentrated extract.

12. The method as claimed in claim 11, wherein the algae are Chlorella, Spirulina, Haematococcus or Dunaliella.

13. A method for manufacturing the composition as claimed in claim 1 comprising:
providing algae; and
preparing the algae to a composition.

14. The method as claimed in claim 13, wherein the algae are in the form of a concentrated extract.

15. The method as claimed in claim 14, wherein the algae are Chlorella, Spirulina, Haematococcus or Dunaliella.

16. A method of eliminating abnormal conditions in a human affected by electromagnetism, comprising
providing a composition containing algae; and
applying the composition to a source generating electromagnetism.

17. The method as claimed in claim 16, wherein the composition was prepared in the form of a gel, a paste, a spray, or immobilized in/into a solid.

18. The method as claimed in claim 17, wherein the composition was a paste.

19. The method as claimed in claim 16, wherein the algae are Chlorella, Spirulina, Haematococcus or Dunaliella.

20. The method as claimed in claim 19, wherein the algae are Dunaliella.
